(19) United States
(12) Patent Application Publication
(10) Pub. No.: US 2026/0060590 A1
(43) Pub. Date: Mar. 5, 2026

(54) COMPUTERIZED METHOD AND SYSTEM FOR DETECTION AND PREDICTION OF CARDIOVASCULAR EVENTS

(71) Applicant: SHEBA IMPACT LTD., Ramat Gan (IL)

(72) Inventors: Michal COHEN-SHELLY, Herzliya (IL); Robert KLEMPFNER, Herzliya (IL); Eran RIVA, Herzliya (IL); Aias MASALHA, Tel Aviv (IL)

(21) Appl. No.: 19/384,026

(22) Filed: Nov. 10, 2025

Related U.S. Application Data
(63) Continuation-in-part of application No. PCT/IL2025/050083, filed on Jan. 23, 2025.
(60) Provisional application No. 63/624,323, filed on Jan. 24, 2024.

(51) Int. Cl. A61B 5/329; A61B 5/00; A61B 5/339; A61B 5/352; A61B 5/366

(52) U.S. Cl. CPC A61B 5/329; A61B 5/339; A61B 5/352; A61B 5/366; A61B 5/4836; A61B 5/7203; A61B 5/7275

(57) ABSTRACT

Provided herein are computer implemented methods and systems for prediction of major adverse cardiovascular events (MACE) and/or detection of CAD in a patient, based on phases of exercise electrocardiogram (ECG) test (EET) data.

### CROSS-REFERENCE TO RELATED PUBLICATIONS

This application is a Continuation-in-Part of International PCT Patent Application No. PCT/IL2025/050083 having International filing date of Jan. 23, 2025, which claims the benefit of priority of U.S. Provisional Patent Application No. 63/624,323, filed Jan. 24, 2024. All of these documents are incorporated by reference in their entirety.

### TECHNICAL FIELD

The present disclosure relates generally to computerized systems and methods for detection of Coronary artery disease (CAD) and/or prediction of major adverse cardiovascular events (MACE) based on exercise ECG Test (EET).

### BACKGROUND

Cardiovascular Diseases (CVDs), including coronary artery disease (CAD), valvular diseases, heart failure, and arrhythmias, are leading causes of global illness and death, impacting hospital admissions and mortality. While substantial resources have been allocated to develop secondary prevention treatments over recent decades, resulting in groundbreaking medications and a range of procedures, progress in primary prevention has been limited. This is particularly true for early diagnosis in those who appear to be healthy.

Coronary artery disease (CAD) being a leading cause of morbidity and mortality worldwide, characterized by the narrowing or blockage of coronary arteries due to atherosclerosis. Early detection is crucial to prevent adverse cardiovascular events.

Current guidelines for primary prevention recommend using risk calculators to measure individual risks for MACE and recommend preventive interventions. Despite the existence of these cardiovascular risk assessment tools for MACE (Framingham Risk Score (FRS), Pooled Cohort Equations (PCE), Systematic Coronary Risk Evaluation (SCORE), and QRISK3), they lack sufficient sensitivity and specificity and have been found to either overestimate or underestimate CVD risk in specific subpopulations.

The Exercise ECG Test (EET), regarded as an outdated but affordable and widely used screening method for CAD, is utilized for asymptomatic individuals as part of routine medical screenings in workplaces, and for those with cardiovascular risk factors or presenting mild or atypical symptoms. The EET records a continuous, 12-lead ECG across three phases: rest, exercise, and recovery. EET data also includes critical measurements like maximal functional capacity via oxygen consumption, heart rate response, and blood pressure changes. However, its simplicity and affordability come with limitations in sensitivity and specificity, leading to the underutilization of much of the recorded data.

Non-invasive tests such as the stress electrocardiogram (ECG) are used for early CAD diagnosis and general screening. During a stress ECG, the heart's electrical activity is monitored under physical exertion to identify ischemic changes indicative of CAD. However, the diagnostic accuracy of stress ECGs varies, with sensitivity ranging from approximately 68% to 77% and specificity from 50% to 77%, potentially leading to false positives or negatives.

Artificial Intelligence (AI) analysis tools have been applied to resting ECG tests using a single 12-lead ECG input, for detecting of several cardiac conditions (Harmon D M et. al., (2022) Cardiovascular Research, Volume 118, Issue 3, February 2022, Pages e23-e25).

Nevertheless, there is a need in the art for computerized systems and methods that can provide enhanced and accurate detection for CAD and for prediction of MACE in patients, wherein the methods are sensitive, specific, accurate, cost effective and can be easily implemented in various health care settings.

### SUMMARY

Aspects of the disclosure, according to some embodiments thereof, relate to advantageous computerized systems and methods for detection or diagnosis of CAD and/or prediction of MACE in a tested patient, based on Exercise ECG Test (EET) performed by the subject.

In some embodiments, there are provided herein advantageous computerized systems and methods for prediction of MACE in a tested patient, based on Exercise ECG Test (EET) performed by the subject.

In some embodiments, there are provided herein computerized systems and methods for detection or diagnosis of CAD in a tested patient, based on Exercise ECG Test (EET) performed by the subject.

According to some embodiments, as detailed herein, the advantageous computerized methods disclosed herein utilize Artificial Intelligence (AI), and specifically Machine Learning (ML), to provide MACE prediction based on EET data (results) of the tested patient. Advantageously, the methods disclosed herein facilitate the use of the vast amount of detailed information/data that can be extracted from an EET, to thereby improve short and long-term prediction of cardiovascular disease and eventually enable better prevention of MACE. As detailed herein, the advantageous methods disclosed herein can facilitate detection or identification of patterns in an image (e.g., EET waveforms) that are otherwise undetectable.

According to some embodiments, as detailed herein, the computerized methods disclosed herein utilize Artificial Intelligence (AI), and specifically Machine Learning (ML), to provide CAD diagnosis and/or MACE prediction based on EET data (results) of the tested patient. Advantageously, the methods disclosed herein facilitate the use of the vast amount of detailed information/data that can be extracted from an EET, to thereby improve detection of CAD and/or MACE.

According to some embodiments, the disclosed models architecture addresses the redundancy in ECG signals by randomly selecting varied segments in each training iteration. Consequently, the model is exposed to a diverse range of signal patterns within individual ECG recordings, significantly enhancing its learning capacity and generalizability. In some embodiments, advantageously, by generating multiple predictions per subject from different ECG segments, the model provides a more robust assessment of cardiac health. Advantageously, this also leads to a stable performance evaluation, as averaging predictions across various segments provides a more reliable indicator of cardiac events, reducing variability and improving the overall accuracy of the model's MACE risk assessments.

Thus, in some embodiments, the methods and systems disclosed herein can provide enhanced prevention and interventions for cardiac related issues such as structural abnormalities, coronary artery disease (CAD), and Major Adverse Cardiovascular Events (MACE). As further exemplified herein, the disclosed methods, based on EET, unexpectedly exhibit enhanced prediction results (as determined for example, by sensitivity, specificity and/or Area under the curve (AUC)), as compared to other tests (for example, using resting ECG).

According to some embodiments, incorporating the ML and AI methods disclosed herein for stress ECG interpretation is advantageous for various reasons: Accuracy enhancement, as the disclosed AI algorithms can detect subtle changes in ECG patterns, increasing sensitivity and specificity in CAD diagnosis and MACE prediction; Early detection, as the enhanced analysis facilitates the identification of CAD at earlier stages, enabling timely intervention; Non-Invasive efficiency, as the AI augments the diagnostic value of stress ECGs without additional invasive procedures, reducing patient risk and discomfort.

According to some embodiments, applying methods disclosed herein, utilizing the AI models with stress ECG (EET) (raw) data, improves the early detection and diagnosis of CAD, leading to better patient outcomes, through timely and accurate identification of the condition.

According to some embodiments, utilizing trained AI models with stress ECG interpretation is advantageous as they can improve accuracy (for example, the AI algorithms can detect subtle changes in ECG patterns, increasing sensitivity and specificity in CAD diagnosis); enable early detection (for example, the enhanced analysis can facilitate the identification of CAD at earlier stages, enabling timely intervention); and augment the diagnostic value of stress ECGs without additional invasive procedures, reducing patient risk and discomfort. According to some embodiments, there is provided a

According to some embodiments, there is provided a computer implemented method for prediction of major adverse cardiovascular events (MACE) and/or detection of coronary artery disease (CAD) in a subject, the method includes:
receiving exercise electrocardiogram (ECG) test (EET) data of the subject;
inputting waveform data from the EET data to a predictive machine learning (ML) algorithm, wherein the machine learning algorithm was trained on a dataset comprising data patterns of R-R-R segments of EET data obtained from a plurality of subjects and correlated with the presence or absence a coronary artery disease in each of said plurality of subjects;
outputting by the machine learning algorithm, a MACE risk score and/or a CAD probability score for the patient, based on the inputted data from the EET data for the subject;
to thereby provide prediction of CAD or MACE in the patient.

According to some embodiments, the raw data is preprocessed to remove noise components, to standardize the data and/or to segment into at least exercise and recovery sections. Each possibility is a separate embodiment.

According to some embodiments, the R-R-R segments includes information regarding momentary heart activity, comprising a complete QRS complex.

According to some embodiments, the EET test may include recording obtained from single-lead, multi-lead, a 12-lead test, or any combinations thereof.

According to some embodiments, the EET test includes a 12-lead test.

According to some embodiments, data obtained from at least a portion of EET leads is utilized for the determination of the R-R-R segments.

According to some embodiments, data obtained from leads V5-V6 may be used for selecting a lead having least variance between R peaks, for the R-R-R segmentation.

According to some embodiments, R-R-R segmentation of R-peaks of QRS complex are determined for at least a portion of selected leads, based on:
determining differences between R-peak locations for each of the selected leads;
calculating variance in R-peak locations across each of the selected leads; and
selecting the lead with the least variance between the peaks for R-R-R segmentation.

According to some embodiments, the method further comprises standardizing length of the R-R-R segments. In some embodiments, the standardization may be part of the preprocessing step.

According to some embodiments, the method may further include applying a classification transformer.

According to some embodiments, the EET data includes raw waveform EET data of at least a portion of (each) of at least three phases of EET. According to some embodiments, the three phases of the EET include a rest phase, a stress phase and a recovery phase.

According to some embodiments, the dataset may be split into a plurality of groups, to enhance balanced representation of cardiac related events, and non-related events.

According to some embodiments, the period of time for MACE prediction is for six months or more, from performing the EET test. In some embodiments, the prediction is for over a year, over 1.5 years, over 2 years, over 5 years or more, from taking the test.

According to some embodiments, the EET data may further include oxygen consumption of the subject, heart rate, blood pressure, or any combinations thereof.

According to some embodiments, the method may further include providing a therapy recommendation to the patient, based on the MACE risk score and/or the CAD probability score output.

According to some embodiments, the therapy may include: a pharmaceutical therapy, behavioral therapy, a surgical therapy, or any combinations thereof.

According to some embodiments, there is provided a system for prediction of MACE and/or detection of coronary artery disease CAD in a subject, the system includes a processor configured to execute the method for prediction of MACE and/or detection of CAD.

According to some embodiments, the system may further include or by communicatively associated with one or more of: an ECG unit, a display, a user interface, a memory, a local server, a remote server, a communication unit, a database, or any combination thereof.

According to some embodiments, there is provided a non-transitory computer-readable medium storing processor executable instructions on a computing device, when executed by a processor, the processor executable instructions causing the processor to perform the method for prediction of MACE and/or detection CAD.

According to some embodiments, there is provided a computer implemented method for prediction of major adverse cardiovascular events (MACE) for a patient, the method includes:
receiving exercise electrocardiogram (ECG) test (EET) data of the patient;
inputting waveform data from the EET data to a predictive machine learning (ML) algorithm, wherein the machine learning algorithm was trained on a dataset including raw waveform data of at least a portion of at least three phases of EET data obtained from a plurality of subjects and correlated with a cardiac related event in each of said plurality of subjects, over a period of time;
outputting by the machine learning algorithm, a MACE risk score for the patient, based on the inputted data from the EET data for the subject; to thereby provide prediction of MACE in the patient.

According to some embodiments, the at least three phases of the EET may include a rest phase, a stress phase and a recovery phase.

According to some embodiments, at least a portion of a phase may include about 8-12 seconds of the phase.

According to some embodiments, at least a portion of a phase comprises about 10 seconds of the phase.

According to some embodiments, a consecutive portion of a rest phase, a stress phase and a recovery phase are utilized for the dataset.

According to some embodiments, a plurality of randomized sub-portions (intervals) of each of the at least three phases of the EET data may be processed.

According to some embodiments, a sub portion may include a time frame of about 1-4 seconds. According to some embodiments, a sub-portion may include a time frame of about 2 seconds.

According to some embodiments, the dataset may be split into a plurality of groups, to enhance balanced representation of cardiac related events and non-related events.

According to some embodiments, raw waveform data may be preprocessed, to obtain a matrix representing voltage over time for each recording lead of the EET.

According to some embodiments, one or more features may be extracted from the EET waveforms.

According to some embodiments, the EET test includes a 12-lead test.

According to some embodiments, waveform data obtained from one or more or each of the leads may be processed.

According to some embodiments, the ML algorithm may include convolutional layers, transformer layers, filters, adaptive optimizers, or combinations thereof.

According to some embodiments, the MACE may include Myocardial Infarction (MI), Stroke (Ischemic or Hemorrhagic), and/or mortality.

According to some embodiments, the MACE may be associated with coronary artery disease (CAD), valvular disease, heart failure, arrhythmias, or any combination thereof.

According to some embodiments, the cardiac related event may include: mortality from a cardiac related event, stroke, cerebral vascular accident (CVA), ischemic heart disease (IHID), Percutaneous coronary intervention (PCI) or combinations thereof.

According to some embodiments, the period of time may be about 2 years.

According to some embodiments, the EET data may further include oxygen consumption of the subject, heart rate, blood pressure, or any combinations thereof.

According to some embodiments, the method may further include providing a therapy recommendation to the patient, based on the output risk score of the patient.

According to some embodiments, the therapy may include: a pharmaceutical therapy, behavioral therapy, a surgical therapy, or any combinations thereof.

According to some embodiments, there is provided a system for prediction of major adverse cardiovascular events (MACE) in a patient, the system includes a processor configured to execute the method disclosed herein.

According to some embodiments, the system may further include or be communicatively associated with one or more of: an ECG unit, a display, a user interface, a memory, a local server, a remote server, a communication unit, a database, or any combination thereof.

According to some embodiments, there is provided a non-transitory computer-readable medium storing processor executable instructions on a computing device, when executed by a processor, the processor executable instructions causing the processor to perform the method disclosed herein.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes.

In the figures:

FIG. 1—shows a flowchart of a method for MACE prediction based on EET data (results), according to some embodiments;

FIG. 2—shows a flowchart of a method for CAD detection and/or MACE prediction based on EET data, according to some embodiments;

FIGS. 3A-B show line graphs and Area under the curve (AUC) of True positive rate vs. False positive rate MACE prediction, based on data from ECG rest part only (FIG. 3A) or data from three phases of ECG test (FIG. 3B);

FIGS. 4A-B show MACE Prediction differences in Receiver Operation Characteristics (ROC), rest-only and all-sections of EET. FIG. 4A—results from AI Model trained with only Rest section of EET; FIG. 4B—results from AI Model trained with Rest, Exercise and Recovery sections of EET;

FIGS. 5A-B show CAD Prediction differences in ROC between rest-only and all-sections of EET. FIG. 5A—results from AI Model trained with only Rest section of EET; FIG. 5B—results from AI Model trained with Rest, Exercise and Recovery sections of EET;

FIGS. 6A-B—line graphs of averaging 100 R-R-R segments from rest ECG. FIG. 6A shows zero-padded segments averaged; FIG. 6B shows averaging after applying cubic spline interpolation.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

According to some embodiments, there are provided herein computer implemented methods and systems, for providing MACE prediction of patient and/or CAD detection, based on exercise electrocardiogram (ECG) test (EET) results of the patient.

The term “CAD” (Coronary Artery Disease) relates to a chronic condition characterized by the narrowing or blockage of coronary arteries due to atherosclerosis (buildup of plaque including cholesterol, fats, and other substances). CAD can lead to reduced blood flow to the heart, increasing the risk of myocardial infarction (heart attack) and other cardiovascular complications. Complications of CAD may include Myocardial infarction (heart attack), heart failure, Arrhythmias, sudden cardiac death, and the like. CAD is a progressive vascular disease that leads to MACE.

The term “MACE” (Major Adverse Cardiovascular Events) refers to a composite of serious cardiovascular events that indicate poor clinical outcomes in patients with cardiovascular disease. Components of MACE include, for example, but not limited to: Myocardial Infarction (MI), acute blockage of coronary arteries leading to heart muscle damage, Stroke (Ischemic or Hemorrhagic), interruption of blood supply to the brain, leading to neurological deficits, cardiovascular death, and the like. Hence, early diagnosis and prediction (along with optional appropriate medical or interventional therapy) can significantly reduce MACE incidence and improve survival.

The term “ECG” (electrocardiogram) relates to a non-invasive diagnostic test that records the electrical activity of the heart over time using electrodes placed on the skin of a subject. Generally, key components of an ECG include: P wave (representing Atrial depolarization (atrial contraction)); PR interval (time taken for electrical impulse to travel from atria to ventricles); QRS complex (representing Ventricular depolarization (ventricular contraction)); ST segment (represents the early phase of ventricular repolarization); ST segment (represents the early phase of ventricular repolarization); T wave (Ventricular repolarization (relaxation phase)); and QT interval (Total time for ventricular depolarization and repolarization).

The term “QRS complex” is part of an ECG waveform and it represents ventricular depolarization, which is the electrical activation of the ventricles leading to their contraction. The QRS appears as a sharp, rapid deflection on an electrocardiogram (ECG). Components of the QRS complex include Q Wave (the first negative (downward) deflection before an R wave, indicating septal depolarization); R Wave (the first positive (upward) deflection, representing ventricular muscle depolarization; and S Wave (a negative deflection following an R wave, representing the late phase of ventricular depolarization).

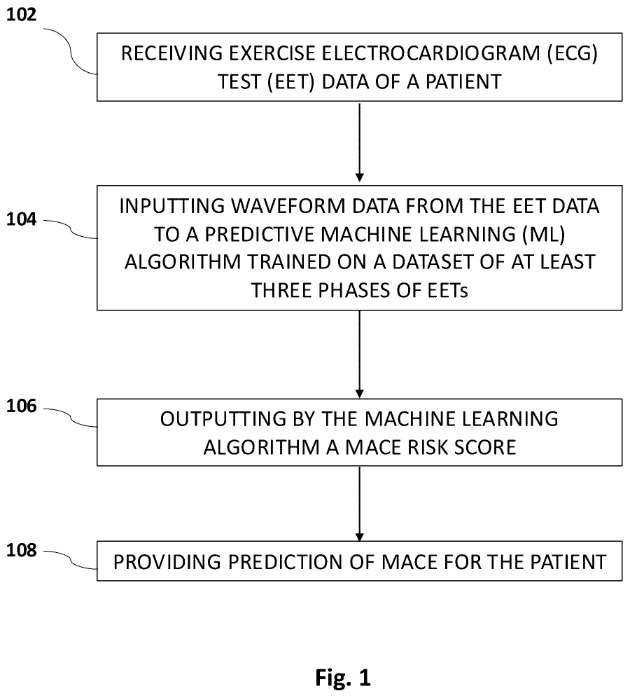

Reference is made to FIG. 1, which shows steps in a computerized method for MACE predictions, according to some embodiments. As shown in FIG. 1, at step 102, exercise electrocardiogram (ECG) test (EET) data (results) of a patient are obtained. The EET test may be performed instantly or may retrieved from previous EET the patient has conducted. The results may be provided as raw data waveform, or in any type of suitable format. In some embodiments, the EET data may be preprocessed to a suitable format. In some exemplary embodiments, the EET is a 12-lead ECG test. Next, at step 104, the data form the EET of the subject is applied to a trained, predictive machine learning (ML) algorithm. As detailed herein, the ML has been trained on a data set which includes raw data waveform of portions (i.e., temporal portions) of at least three phases (for example, rest phase, stress phase and recovery phase) of EET test performed by a plurality of subjects and correlated with respective cardiovascular events of the subjects. As detailed herein, the data set may be from a plurality of subjects (preferably including subjects of different age, gender, medical history, health condition, etc.). Next, at step 106, based on the data form the EET of the patient, the ML algorithm outputs/calculates a MACE risk score for the subject. At step 108, based on the outputted/calculated risk score, MACE prediction to the patient is provided. In some embodiments, as detailed below, the prediction is for X-period of time (for example, 1-5 years) after the completion of the EET.

According to some embodiments, the temporal portions of the phases of the EET may be in the range of about 4-30 seconds, or any subranges thereof, such as, for example, about 5-18 seconds, about 6-16 seconds, about 7-14 seconds, about 8-12 seconds, about 10 seconds. In some embodiments, the temporal portion for each phase is similar of different between the phases.

In some embodiments, the temporal portions may be randomly divided into sub-portions, such as, for example, in then length of 1-10 seconds, 1.5-6 seconds, 2-4 seconds, and the like, wherein a plurality of such sub portions may be included for the training of the algorithms.

According to some embodiments, the at least three phases/stages of the EET include a rest phase (stage), a peak (stress) stage and a recovery stage. In some embodiments, consecutive portions of the phases may be used for training and/or execution of the ML algorithm.

According to some embodiments, for training the model, retrospective EET data (for example, obtained from electronic medical records (EMRs) of a health care facility) may be used for creation of the training data set. According to some embodiments, the outcome of the predictive model may include the patient's occurrence of MACE (Major Adverse Cardiovascular Event) within a period of time from the Exercise ECG test (EET), such as, for example, but not limited to: 1-10 years, 2-5 years, 2 years from the EET timing, and the like. In some embodiments, as event may be considered as one of more of: IHD (Ischemic heart Disease), CVA (cerebral vascular accident), PCI (Percutaneous coronary intervention), stroke, Mortality, and the like, or any combinations thereof.

In some embodiments, the ECGs may be acquired as digital standard 12-lead ECGs. In some embodiments, a 12-lead ECG configuration includes Limb Leads: I, II, III, aVR, aVL, aVF; Precordial (Chest) Leads: V1, V2, V3, V4, V5, V6. Each lead represents the electrical potential difference between two points (bipolar) or relative to a reference point (unipolar). In some embodiments, leads refer to the electrical signals derived from the electrodes.

In some embodiments, for the training, binary classification using the 12-leads ECG recording and/or sub-group analysis by age and sex may also be performed.

According to some embodiments, the ECG data may be preprocessed for use as training set and/or for use as input to the ML algorithm. In some embodiments, raw ECG waveform data may be extracted from rdt files, which may be converted into matrix (for example, numpy format), wherein each cell in the matrix may represent voltage over time. In some embodiments, for each test, a table which contains 12 columns (representing the 12 leads) and X amount of rows (based on the recording timing) is formed. In some embodiments, for each sample, quality checks for constancy and completeness may be performed.

In some embodiments, the ECG data may be segmented into at least distinct phases (stages), including, rest phase, peak exercise phase, and recovery phase, while selecting specific time windows (temporal portions) for analysis, for each phase. Each such segment may then be saved as a numpy file (files used for storing arrays in NumPy) for further processing.

According to some embodiments, and without wishing to be bound to any theory or mechanism, ECG data segmentation into rest, peak exercise, and recovery phases may have clinical rationale. The rest phase establishes baseline cardiac function, key for spotting irregularities. The peak exercise phase, when the heart is under maximum stress, uncovers potential ischemic issues not visible at rest. The recovery phase assessment is important for evaluating the heart's return to baseline, providing insights into overall cardiac health. This targeted analysis across different stress levels on the heart is important for a comprehensive cardiovascular assessment, further facilitating the prediction of major adverse events.

According to some embodiments, a custom function may be applied, to split the dataset into several folds, thereby providing balanced representation of “event” and “non-event” cases. In some embodiments, this step may include options for random shuffling and oversampling of minority classes. In some embodiments, the processed data may be merged with the fold information. According to some embodiments, such preprocessing may ensure the uniformity, integrity and usability of the ECG data for use with the disclosed algorithm.

According to some embodiments, the disclosed algorithm may include a deep learning framework including convolutional and transformer layers. In some embodiments, the convolutional layers may extract features from ECG signals, and the transformer, may be used to capture sequential dependencies. In some embodiments, data loaders that handle ECG data in stages (rest, exercise, recovery) may apply filters and normalization (for example, by standard deviation). In some embodiments, training of the model may involve a customized loss function, with a Y-second window for ECG signals. In some embodiments, the model may use an adaptive optimizer with specific learning rate adjustments, and the training may include early stopping based on AUC-ROC performance, or other desired parameters.

According to some embodiments, the model's architecture may employ a distinctive Y-second interval (sub-portion) in the ECG data processing, thereby efficiently capturing essential cardiac signatures. This can address the redundancy in ECG signals by randomly selecting varied segments in each training iteration. Such intervals are as informative as longer recordings. Consequently, the model is exposed to a diverse range of signal patterns within individual ECG recordings, significantly enhancing its learning capacity and generalizability. In some embodiments, the Y-second interval approach not only enhances training efficiency but also stabilizes performance during prediction. In some embodiments, by generating multiple predictions per subject from different ECG segments, the model provides a more robust assessment of cardiac health. Advantageously, this leads to a stable performance evaluation, as averaging predictions across various segments provides a more reliable indicator of cardiac events, reducing variability and improving the overall accuracy of the model's MACE risk assessments.

According to some embodiments, the output risk score is indicative of MACE prediction. In some embodiments, the score may be any value in the range of between 0-1, or any other corresponding numerical scale.

In some embodiments, the MACE prediction is for X time after the EET test. For example, the X time may be 1-6 months, 0.5, 1, 2, 3, 4, 5, 6, 7, 8, 9, 10 years or more after the EET. Each possibility is a separate embodiment.

According to some embodiments, based on the predicted MACE for the subject, a therapy management may be provided or recommended to the subject. Such therapy may include, for example, but not limited to: a pharmaceutical therapy (e.g., a medicament), behavioral therapy (e.g., diet change or physical activity), a surgical therapy (e.g., surgery, stent insertion), and the like, or any combinations thereof.

According to some embodiments, the ML is configured to identify/determine/associate/classify one or more ECG waveform parameters, values or patterns obtained from at least three phases of the EET, with cardiac event risk.

According to some embodiments, stress ECG tests provide a challenge for machine learning models that rely on fixed-length standardized input. The length of the exercise portion of the test can range anywhere from 10 seconds up to 30 minutes to reach target heart rate that is adjusted based on age as is common in Bruce Protocol. Moreover, signal noise patterns increase with strain due to muscle activity, breathing, sweat and electrode movement. To overcome these issues, a preprocessing stage of the results may be applied to each recording.

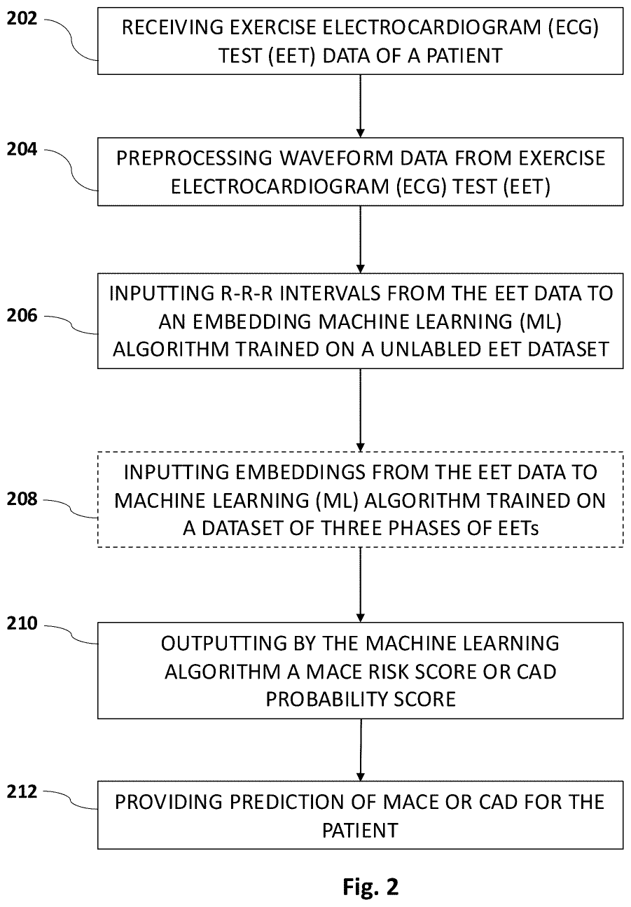

Reference is made to FIG. 2, which shows steps in a computerized method for MACE prediction and/or CAD detection, according to some embodiments. As shown in FIG. 2, at step 202, exercise electrocardiogram (ECG) test (EET) data (results) of a patient are obtained. The EET test may be performed instantly or may retrieved from previous EET the patient has conducted. The results may be provided as raw data waveform, or in any type of suitable format. In some embodiments, the EET data may be preprocessed to a suitable format. In some exemplary embodiments, the EET is a 12-lead ECG test. Next, at step 204, waveform data from the EET is preprocessed. In some embodiments, the preprocessing may include filtering noise, standardization and/or segmentation (for example, to exercise and recovery sections of the test), as detailed herein. At step 206, R-R-R intervals from the EET data are applied to an embedding machine learning (ML) algorithm, trained on an unlabeled EET dataset. As detailed herein, the dataset may include data from a plurality of subjects that have undergone EET test, preferably including subjects of different age, gender, medical history, health condition, etc.). optionally, at step 208, embeddings from the EET data is applied to machine learning (ML) algorithm trained on a dataset of three phases of EETs. Next, at step 210, based on the data form the EET of the patient, the ML algorithm outputs/calculates a MACE risk score or CAD probability score for the subject. At step 212, based on the outputted/calculated risk score, MACE or prediction and/or CAD diagnosis/detection is provided to the patient.

According to some embodiments, for the preprocessing, Raw files containing integers (for example, 4-digits integers), including measured voltage levels in 12-Lead ECG (I, II, III, aVR, aVF, aVL, V1, V2, V3, V4, V5, V6) are converted into 32-bit floating-point arrays. These may be divided by a factor of 1,000, to be in the micro-volt scale. In some embodiments, only selected leads (for example, 8, such as, aVR, aVF, V1, V2, V3, V4, V5, V6) may be used. In order to remove noise components (such as, baseline drift, breathing and muscle activity), a filter (such as, A 7-th order Butterworth 1-30 Hz Bandpass filter) may be applied. Optionally, the signal may be decimated to a desired sampling rate (for example, decimating from a recorded 500 Hz sampling rate, by a factor of five to bring the resulting sampling rate to 100 Hz). Each recording may be segmented into two sections—exercise and recovery, where the exercise segment is defined by a Bruce Protocol.

According to some embodiments, in order to generate a more standardized input for the machine learning models, each patient's naturally forming segments that vary with heart rate may be utilized. To this aim, R-peaks of the ECG QRS complex are identified, for example, along V3-V6 or V5-V6 peaks. The difference between R-peak locations may further be used to calculate the variance across each of the selected leads (for example, V3, V4, V5 and/or V6). The lead with the least variance is considered the most consistent and is used for a “R-R-R” segmentation across all other leads. It is noted that R-R-R segments include all necessary information about momentary heart activity, including a complete QRS complex in the middle, P-wave, T-wave and other potential cardiac electrical activity. Each segment begins and ends in R-peaks of adjacent QRS complexes. As each segment length varies, an interpolation (such as, a cubic spline interpolation) may be used, to stretch the input segment to standardized samples (for example, 250 samples, corresponding to 2.5 seconds), in cases where no interpolation is necessary in low heart rates. This advantageously allows to more accurately compare ECG components between various heart beats. In some embodiments, each input recording may be saved as two filed containing point data after preprocessing (for example, parquet files containing 16-bit floating point data). The shape of the output file may be determined, as (Y1*N, Y2), where Y1 stems from interpolation parameters, Y2 is the number of leads after reduction and N is the number of R-R-R segments in the signal. For example, the shape of the output file may be determined as (250*N, 8) where 250 stems from interpolation parameter, 8 is the number of leads after reduction and N is the amount of R-R-R segments found in the signal. Each of the three recordings corresponds with exercise and recovery EET sections.

According to some embodiments, in order to reduce the need for a large scale of input samples to learn patterns that separate between desired groups (for example, healthy vs. pathological), an unsupervised Deep-Learning model may be employed. An exemplary unsupervised model may include a Maximum Mean Discrepancy Variational AutoEncoder (MMD VAE)). Such model(s) may be employed, to learn/train on the patterns of R-R-R segments, and compress each segment using, for example, a Gaussian latent space. Accordingly, a dataset of preprocessed samples may be used. The dataset may be split as desired between training/validation/test sets (for example, as 60%/20%/20%, respectively). The split may be stratified with respect to various patient characteristics, such as, age (for example, 0-40, 40-60, 60-80, 80-100 bins) and gender. The Latent space dimension y2 per each file inputted, the output is (N, y2), where N is the number of segments in the file. Training may be shuffled along segments, without time consideration as to allow time-independent learning of representation. In some examples, with the Maximum Mean Discrepancy (MMD) loss, the latent space is a set of 48 Gaussian distributions. In some embodiments, the pre-processing utilize an unsupervised ML model.

In some embodiments, the unsupervised Deep-Learning model may be evaluated on a a validation set, and the epoch having the lowest validation loss may be selected, while its corresponding weights may be used in inference. In some embodiments, selection bias against subjects that routinely (with no prior suspicion of morbidity) perform a stress ECG test may be avoided. To this aim, in the heart mapping dataset the negative (healthy) label were randomly taken from a larger stress ECG dataset, by age and gender matching to the population that received negative label, after performing a heart mapping. Accordingly, this would results in a dataset of A1 healthy matched recordings against A2 afflicted matched recordings that received positive label in the heart mapping test, indicating presence of CAD. In some embodiments, a split (for example 60%/20%/20%) may be performed.

In some embodiments, the recordings may undergo inference, for example, using the MMD VAE model, and its input may be inserted to a next model which is a classification transformer. Each input to the transformer consisted of (N, y2) values, where N is the variable number of exercise and recovery segments were extracted from the recording, and y2 (for example, 48) is the aforementioned latent space.

According to some embodiments, the data is preprocessed and segmented, using R-peaks, forming R-R-R intervals. The R-R-R intervals may be fed to a suitable model, such as Variational Autoencoder (VAE) model. According to some embodiments, models that receive as input the VAE embeddings and output an individual's risk score for 2-Y cardiovascular event (Binary classification model) and probability for existing myocardial ischemia are trained. The Model may be assessed using performance metrics, such as, area under the receiver operating characteristic curve (AUROC), sensitivity, specificity, accuracy, positive predictive value (PPV), and negative predictive value (NPV). In some embodiments, for MACE prediction, each sample may be labeled as 0=no event or 1=event that occurred within 2-Y, event considered as mortality or stroke or ischemic heart disease, event being considered as mortality or stroke or ischemic heart disease. In some embodiments, for CAD, that samples may be labeled as 0=myocardial ischemia under 5% (<5) and 1=over 5% (>5).

According to some embodiments, for preprocessing, raw waveform undergoes a few stages to reduce noise levels and provide uniformity intra-patient and inter-patient. Data provided as integer is converted into floating-point numbers and divided by 1,000 to bring it to known scales. In some cases, only some of the leads (for example, leads aVR, aVF, V1-V6) may be used to reduce data redundancy. A 7^{th} order Butterworth Bandpass Filter 1-30 Hz may be applied twice (back and forth) to reduce motion, breathing, sweat, muscles and powerline noises. Signal is decimated by a factor of 5 to reduce output sizes and segmented into R-R-R intervals using V5/V6 R-peak detection. Resultant intervals may then be interpolated (for example, using cubic spline) to 2.5 seconds (on 100 Hz, equals 250 sample points) as to provide consistency between beats of variable heart rates. Data may be saved as 16-point float using a Gzip-compressed parquet file.

According to some embodiments, a custom function to split the dataset into several folds or training/validation/testing split may be used, to ensure balanced representation of event and non-event cases by use of label-gender-age bin stratification (age bins may be 0-40, 40-60, 60-80, 80-100). This step includes options for random shuffling. Thereafter, the processed data may be merged with fold information. This detailed preprocessing ensures the uniformity, integrity and usability of the ECG data for use with the AI-driven algorithm.

According to some embodiments, in training, the dataset may be batched into shuffled sets of inputs (for example, 32 inputs), pad them to largest sequence and mask them accordingly. Each sequence may be preceded by a classification token used to output classification prediction. The classification output may be then be fed into two fully-connected layers, where the last output is of size one. Thus, output closer to zero (o) denotes healthy prediction and output of one (1) is that of CAD prediction in heart mapping test. A function (Such as, a sigmoid function) is then used to transform the output to a probability space, which is fed into a focal loss function. The loss function may be with alpha=0.25 to account for class imbalance. The best model may be selected using the lowest validation loss. Threshold for binary prediction may be calculated, for example, using maximal F1-score across all possible thresholds in the validation dataset.

According to some embodiments, a Maximum Mean Discrepancy Variational Autoencoder (MMD-VAE or InfoVAE) deep learning foundational model that takes R-R-R segments and outputs embeddings of 48 latent dimensions based using MMD and Mean Squared Error losses may be utilized. The Embeddings are then fed into transformer-encoder model using a learned/trained rest/exercise/recovery section embedding. A fixed class token may be added prior to the embeddings for classification. The transformer block may use 4 encoders, 2 attention heads and 64 feed-forward dimension. The transformer block output for the classification token may be fed into a 16-dimension fully connected layer with a ReLU activation and then to a 1-dimension output fully connected layer. Loss, such as, is a Sigmoid Focal Loss with alpha=0.1, may be used to assist with class imbalance. Optimizer may further be used, such as, AdamW with AMSGrad and Weight Decay of 0.2, at learning rate 0.0001. According to some embodiments, for the training, validation set may be evaluated at end of each epoch, and early stop may be defined as 30 epochs with no improvement to validation loss. Evaluations may be performed based on model weights with lowest validation loss.

According to some embodiments, data may be fed in the form of embeddings with a variable length due to different recording lengths. A linear regression may be performed for each embedding dimension over a section. If the regression is found significant (p-value<0.05), residuals may be calculated and Z-scored. Residual embeddings over 2SD may be discarded to improve consistency and reduce noise. If the regression was not significant, the slope may be assumed to be zero, and only Z-scoring may be performed prior to >2SD embeddings outlier discarding. Alongside the embeddings, an input mask may be given to denote variable recording lengths, that may be fed into the attention blocks for improved performance and accuracy. Target may be adjusted/changed between MACE and CAD based on the dataset, producing different model weights.

According to some embodiments, an output probability score is indicative of CAD diagnosis prediction. In some embodiments, the score may be any value in the range of between 0-1, or any other corresponding numerical scale. In some embodiments, the score may be binary, wherein 0 is healthy (with respect of CAD) and 1 is sick/afflicted (i.e., having CAD).

According to some embodiments, based on the identification of CAD in the subject, a corresponding therapy management may be provided or recommended to the subject. Such therapies may include, for example lifestyle Modifications (such as, diet, exercise, smoking cessation), Medications (such as, Aspirin, statins, beta-blockers, ACE inhibitors, nitrates, etc.) and/or Procedures (such as, PCI (Percutaneous Coronary Intervention, i.e., stenting), CABG (Coronary Artery Bypass Grafting), etc.).

According to some embodiments, the algorithms disclosed herein may be implemented using an artificial neural network (ANN), such as a deep neural network (DNN), a convolutional neural network (CNN), a recurrent neural network (RNN) and the like, decision trees or graphs, association rule learning, support vector machines, inductive logic programming, Bayesian networks, instance-based learning, manifold learning, sub-space learning, and the like, or any combination thereof. The algorithm or model may be generated using machine learning tools, data wrangling tools, deep learning tools, and, more generally, data science and artificial intelligence (AI) learning tools, as elaborated hereinbelow.]

In some embodiments, the algorithms disclosed herein are executed using one or more processing units that may be further configured to extract features from the EET data, using techniques such as, but not limited to, Principal Components Analysis (PCA), Independent Component Analysis (ICA), Linear Discriminant Analysis (LDA), Locally Linear Embedding (LLE), t-distributed Stochastic Neighbor Embedding (t-SNE), Unified Manifold Approximation and Projection (UMAP) and/or Autoencoders, and the like.

According to some embodiments, the training process may be executed by a suitable training module.

In some embodiments, the training stage of the algorithm may be an offline process, during which a database of training data is assembled and used for the creation of the ML data-analysis model(s)/algorithm(s). In some embodiments, the training stage may be performed “online”.

According to some embodiments, the validation of the models may include evaluation of different model performance metrics, such as, for example, but not limited to: sensitivity, specificity, Aera under the curve (AUC), AUC-ROC, accuracy, precision, recall, F1 score, and the like, or any combinations thereof.

According to some embodiments, as more EET data is collected, the training database may grow in size and may be updated. The updated database may then be used to re-train the algorithm, thereby updating/enhancing/improving the algorithm for MACE prediction output and/or CAD diagnosis. In some embodiments, the new instances in the training database may be obtained from new tested subjects or from previous EET tests that have not been previously used for training.

In some embodiments, the EET test may be performed using any form of electrocardiographic (ECG) or equivalent cardiac electrical activity measurement system. The ECG data may be obtained from any number of leads, including but not limited to single-lead, multi-lead, conventional 12-lead configurations, and the like. The ECG acquisition may further be carried out using various devices, including, for example, but not limited to: wearable devices, patch-based electrodes, chest-strap sensors, other portable or ambulatory ECG monitoring systems, or any combinations thereof.

In some embodiments, the electrocardiographic (ECG) or equivalent cardiac electrical activity data utilized in the EET test may be obtained from systems employing any number of leads or signal acquisition configurations. The ECG data may be derived, for example, from single-lead, dual-lead, multi-lead, 3-lead, 5-lead, 6-lead, 12-lead, 15-lead, or 18-lead systems, or from other configurations suitable for capturing cardiac electrical activity. In some embodiments, high-density or body-surface mapping systems incorporating 32 to 256 or more electrodes may be employed to provide spatially resolved cardiac signal data. The ECG recordings may be acquired using conventional electrode placements, or by means of wearable, patch-based, or chest-strap sensors designed for ambulatory or continuous monitoring.

In some embodiments, the ECG data may be collected using implantable sensors or remote or contactless sensing technologies capable of detecting cardiac electrical or electrophysiological signals.

According to some embodiments, the systems and methods described herein are not limited to any specific ECG hardware configuration or signal acquisition modality, and may encompass all technologies capable of acquiring data representative of cardiac electrical activity suitable for performing the EET test.

In some embodiments, an identified shift in the collected data's distribution may serve as a trigger for the re-training of the algorithm. In other embodiments, an identified shift in the deployed algorithm's performance may serve as a trigger for the re-training of the algorithm. In some embodiments, the training database may be a centralized database (for example, a cloud-based database), or it may be a local database (for example, for a specific healthcare facility). In some embodiments, learning and updating may be performed continuously or periodically on a remote location (for example, a cloud server), which may be shared among various users (for example, between various institutions). In some embodiments, learning and updating may be performed continuously or periodically. In some embodiments, federated learning may be used to update a local model with a model that has been trained on data from multiple facilities/subjects without requiring the local data to leave the facility or the institution.

According to some embodiments, the ML model may be a supervised deep-learning model or an unsupervised deep learning model. In some embodiments, the method may include a combination of supervised and/or unsupervised ML models.

The present invention may be a system, a method, and/or a computer program product. The computer program product may include a computer readable storage medium (or media) having computer readable program instructions thereon for causing a processor to carry out aspects of the present invention.

In some embodiments, the term “model”, “algorithm”, “data-analysis algorithm” and “data-based algorithm” may be used interchangeably.

A computer program (also referred to as a program, software, software application, script or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A computer program can be stored in a portion of a file that holds other programs or data, in a single file dedicated to the program in question, or in multiple coordinated files (for example, files that store one or more modules, sub programs or portions of code). A computer program can be deployed to be executed on one computer or on multiple computers that are located at one site or distributed across multiple sites and interconnected by a communication network.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, for example, JavaScript, Smalltalk, C, C++, TypeScript, Python and R.

The computer readable program instructions may execute entirely on the user's computer, partly on the user's computer, as a stand-alone software package, partly on the user's computer and partly on a remote computer or entirely on the remote computer or server (such as, a cloud based). In the latter scenario, the remote computer (or cloud) may be connected to the user's computer through any type of network, including a local area network (LAN) or a wide area network (WAN), or the connection may be made to an external computer (for example, through the Internet using an Internet Service Provider) including wired or wireless connection (such as, for example, Wi-Fi, BT, mobile, and the like). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention. Moreover, a computer can be embedded in another device, for example, a mobile phone, a tablet, a personal digital assistant (PDA, or a portable storage device (for example, a USB flash drive). Devices suitable for storing computer program instructions and data include all forms of non-volatile memory, media and memory devices, including semiconductor memory devices, for example, EPROM, EEPROM, random access memories (RAMs), including SRAM, DRAM, embedded DRAM (eDRAM) and Hybrid Memory Cube (HMC), and flash memory devices; magnetic discs, for example, internal hard discs or removable discs; magneto optical discs; read-only memories (ROMs), including CD-ROM and DVD-ROM discs; solid state drives (SSDs); and cloud-based storage. The processor and the memory can be supplemented by, or incorporated in, special purpose logic circuitry.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The processes and logic flows described herein may be performed in whole or in part in a cloud computing environment. For example, some or all of a given disclosed process may be executed by a secure cloud-based system comprised of co-located and/or geographically distributed server systems. The term “cloud computing” is generally used to describe a computing model which enables on-demand access to a shared pool of computing resources, such as computer networks, servers, software applications, and services, and which allows for rapid provisioning and release of resources with minimal management effort or service provider interaction.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

The term “exemplary” with respect of specific figures of embodiments, refers to “an example”.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

### EXAMPLES

### Example 1—MACE Prediction Based on EET of a Patient Using a Trained Machine Learning Algorithm

A retrospective study was conducted based on electronic medical records (EMRs) from a large tertiary healthcare center. The outcome of the study included the patient's occurrence of MACE (Major Adverse Cardiovascular Event) within 2 years from the EET (Exercise ECG Test) timing. Event was considered as one of: IHD (Ischemic heart Disease), CVA (cerebral vascular accident), PCI (Percutaneous coronary intervention) or Mortality. Another was existence of moderate to severe myocardial ischemia, defined as over 5% myocardial ischemia as viewed in a SPECT-based Heart Mapping test.

All subjects over 18 years old were included which had an available digital 12 lead Exercise ECG performed in the institution between 2017 and 2023 to create the dataset. Demographics of the subjects shown in Table 1. Subjects missing measurements in the EET report or with incomplete or corrupted ECG waveforms were excluded from the study. All ECGs were acquired as digital standard 12-lead ECGs using Norav Medical ECG devices. The final cohort was divided into 3 subsets, using outcome-stratified random sampling for a cross-validation strategy.

The primary analysis was binary classification using the 12-leads ECG recording, sub-group analysis by age and sex also was performed.

TABLE 1
cohort characteristics

| | | Overall | No Event | Mace within | |
|---|---|---|---|---|---|
| | | (n = 32,693) | (n = 32,129) | 2 Y (n = 564) | P-Value |
| Age, median [Q1, Q3] | | 54.0 | 54.0 | 70.0 | <0.001 |
| | | [46.0, 65.0] | [46.0, 65.0] | [62.0, 76.0] | |
| Sex (Male = 0; | 0 | 22427 (68.6) | 21933 (68.3) | 494 (87.6) | <0.001 |
| Female = 1), n (%) | 1 | 10266 (31.4) | 10196 (31.7) | 70 (12.4) | |
| SMOKER, n (%) | 0 | 28646 (87.6) | 28150 (87.6) | 496 (87.9) | 0.865 |
| | 1 | 4047 (12.4) | 3979 (12.4) | 68 (12.1) | |
| ALCOHOL, n (%) | 0 | 32646 (99.9) | 32082 (99.9) | 564 (100.0) | 1.000 |
| | 1 | 47 (0.1) | 47 (0.1) | | |
| DRUG, n (%) | 0 | 32675 (99.9) | 32111 (99.9) | 564 (100.0) | 1.000 |
| | 1 | 18 (0.1) | 18 (0.1) | | |
| MaxSystolicBP, median | | 170.0 | 170.0 | 168.5 | 0.843 |
| [Q1, Q3] | | [150.0, 180.0] | [150.0, 180.0] | [150.0, 180.0] | |
| MaxDiastolicBP, | | 80.0 | 80.0 | 80.0 | 0.176 |
| median [Q1, Q3] | | [75.0, 80.0] | [75.0, 80.0] | [80.0, 80.0] | |
| MaxHeartRate, median | | 162.0 | 162.0 | 142.0 | <0.001 |
| [Q1, Q3] | | [148.0, 172.0] | [149.0, 173.0] | [123.0, 157.0] | |
| PeakExHR, median | | 160.0 | 160.0 | 137.0 | <0.001 |
| [Q1, Q3] | | [144.0, 170.0] | [145.0, 170.0] | [115.0, 152.0] | |
| PeakExMets, median | | 11.3 | 11.3 | 9.1 | <0.001 |
| [Q1, Q3] | | [9.1, 13.5] | [9.1, 13.5] | [6.2, 11.3] | |
| MaxPredictedHR, | | 168.0 | 168.0 | 153.0 | <0.001 |
| median [Q1, Q3] | | [157.0, 176.0] | [157.0, 176.0] | [146.0, 160.2] | |
| PreTestDuration (min), | | 2.0 | 1.9 | 3.0 | <0.001 |
| median [Q1, Q3] | | [1.0, 3.7] | [1.0, 3.7] | [1.3, 5.8] | |
| ExerciseDuration | | 10.0 | 10.0 | 8.7 | <0.001 |
| (min), median [Q1, Q3] | | [8.1, 12.0] | [8.2, 12.0] | [6.0, 10.5] | |
| RecoveryDuration | | 5.1 | 5.1 | 5.1 | 0.769 |
| (min), median [Q1, Q3] | | [4.5, 5.7] | [4.5, 5.7] | [4.4, 5.8] | |
| DM, n (%) | 0 | 29520 (90.3) | 29013 (90.3) | 507 (89.9) | 0.800 |
| | 1 | 3173 (9.7) | 3116 (9.7) | 57 (10.1) | |
| HTN, n (%) | 0 | 24889 (76.1) | 24450 (76.1) | 439 (77.8) | 0.363 |
| | 1 | 7804 (23.9) | 7679 (23.9) | 125 (22.2) | |
| DEPRESS, n (%) | 0 | 32423 (99.2) | 31866 (99.2) | 557 (98.8) | 0.239 |
| | 1 | 270 (0.8) | 263 (0.8) | 7 (1.2) | |
| RENAL FAILURE, n | 0 | 31947 (97.7) | 31400 (97.7) | 547 (97.0) | 0.302 |
| (%) | 1 | 746 (2.3) | 729 (2.3) | 17 (3.0) | |
| AORT ANEURYSM, n | 0 | 32396 (99.1) | 31840 (99.1) | 556 (98.6) | 0.287 |
| (%) | 1 | 297 (0.9) | 289 (0.9) | 8 (1.4) | |
| CVA, n (%) | 0 | 31811 (97.3) | 31262 (97.3) | 549 (97.3) | 0.941 |
| | 1 | 882 (2.7) | 867 (2.7) | 15 (2.7) | |
| MI, n (%) | 0 | 30212 (92.4) | 29697 (92.4) | 515 (91.3) | 0.361 |
| | 1 | 2481 (7.6) | 2432 (7.6) | 49 (8.7) | |
| STEMI, n (%) | 0 | 32240 (98.6) | 31687 (98.6) | 553 (98.0) | 0.329 |
| | 1 | 453 (1.4) | 442 (1.4) | 11 (2.0) | |
| NON-STEMI, n (%) | 0 | 31706 (97.0) | 31162 (97.0) | 544 (96.5) | 0.539 |
| | 1 | 987 (3.0) | 967 (3.0) | 20 (3.5) | |
| IHD, n (%) | 0 | 27808 (85.1) | 27332 (85.1) | 476 (84.4) | 0.701 |
| | 1 | 4885 (14.9) | 4797 (14.9) | 88 (15.6) | |
| CABG, n (%) | 0 | 31634 (96.8) | 31093 (96.8) | 541 (95.9) | 0.310 |
| | 1 | 1059 (3.2) | 1036 (3.2) | 23 (4.1) | |
| PCI, n (%) | 0 | 29952 (91.6) | 29439 (91.6) | 513 (91.0) | 0.622 |
| | 1 | 2741 (8.4) | 2690 (8.4) | 51 (9.0) | |
| CHF, n (%) | 0 | 31883 (97.5) | 31334 (97.5) | 549 (97.3) | 0.886 |
| | 1 | 810 (2.5) | 795 (2.5) | 15 (2.7) | |
| ATRIAL | 0 | 31269 (95.6) | 30726 (95.6) | 543 (96.3) | 0.523 |
| FIBRILLATION, n | 1 | 1424 (4.4) | 1403 (4.4) | 21 (3.7) | |
| (%) | | | | | |
| ATRIAL FLUTTER, n | 0 | 32404 (99.1) | 31845 (99.1) | 559 (99.1) | 1.000 |
| (%) | 1 | 289 (0.9) | 284 (0.9) | 5 (0.9) | |
| MYOCARDITIS, n | 0 | 32564 (99.6) | 32003 (99.6) | 561 (99.5) | 0.490 |
| (%) | 1 | 129 (0.4) | 126 (0.4) | 3 (0.5) | |
| CARDIOMYOPATHY, | 0 | 30975 (94.7) | 30441 (94.7) | 534 (94.7) | 0.979 |
| n (%) | 1 | 1718 (5.3) | 1688 (5.3) | 30 (5.3) | |
| VALVE REPLACE, n | 0 | 32148 (98.3) | 31593 (98.3) | 555 (98.4) | 0.974 |
| (%) | 1 | 545 (1.7) | 536 (1.7) | 9 (1.6) | |
| AVR, n (%) | 0 | 31963 (97.8) | 31413 (97.8) | 550 (97.5) | 0.794 |
| | 1 | 730 (2.2) | 716 (2.2) | 14 (2.5) | |
| AORTIC SURGERY, n | 0 | 32675 (99.9) | 32112 (99.9) | 563 (99.8) | 0.269 |
| (%) | 1 | 18 (0.1) | 17 (0.1) | 1 (0.2) | |
| HEART | 0 | 32672 (99.9) | 32108 (99.9) | 564 (100.0) | 1.000 |
| TRANSPLANT, n (%) | 1 | 21 (0.1) | 21 (0.1) | | |
| TAVI, n (%) | 0 | 32622 (99.8) | 32059 (99.8) | 563 (99.8) | 1.000 |
| | 1 | 71 (0.2) | 70 (0.2) | 1 (0.2) | |
| PACEMAKER, n (%) | 0 | 32137 (98.3) | 31588 (98.3) | 549 (97.3) | 0.107 |
| | 1 | 556 (1.7) | 541 (1.7) | 15 (2.7) | |
| COPD, n (%) | 0 | 32343 (98.9) | 31793 (99.0) | 550 (97.5) | 0.002 |
| | 1 | 350 (1.1) | 336 (1.0) | 14 (2.5) | |
| RENAL DIS | 0 | 31535 (96.5) | 30992 (96.5) | 543 (96.3) | 0.904 |
| CHRONIC, n (%) | 1 | 1158 (3.5) | 1137 (3.5) | 21 (3.7) | |
| RENAL DIS, n (%) | 0 | 31535 (96.5) | 30992 (96.5) | 543 (96.3) | 0.904 |
| | 1 | 1158 (3.5) | 1137 (3.5) | 21 (3.7) | |
| ATRIAL | 0 | 31104 (95.1) | 30563 (95.1) | 541 (95.9) | 0.440 |
| ARRHYTHMIAS, n | 1 | 1589 (4.9) | 1566 (4.9) | 23 (4.1) | |
| (%) | | | | | |

The primary analysis included binary classification using the 12-leads ECG recording, sub-group analysis by age and sex also was performed.

### Overview of the AT Model

### Data Pre-Processing

In the preprocessing phase, raw ECG waveform data extracted from rdt files (Norav raw ECG data format), which are then converted into matrix (numpy format), each cell in the matrix represents voltage over time. Eventually a csv file (table) created for each test contain 12 columns representing the 12 leads and X amount of rows based on the recording timing, for example 10 seconds will contain 5000 rows since the sample rate is 500. For each sample, quality checks for constancy and completeness are performed. The ECG data is segmented into distinct phases: rest phase, peak exercise phase, and recovery phase. In some instances, selecting specific time windows for analysis, for example, 10 seconds for each phase. Each segment is then saved as a numpy file for further processing.

Next, a custom function is applied, to split the dataset into several folds, ensuring balanced representation of event and non-event cases. This step includes options for random shuffling and oversampling of minority classes. Finally, the script merges the processed data with fold information. The cohort was split into 3 folds for cross validation strategy.

This detailed preprocessing ensures the uniformity, integrity and usability of the ECG data for use in the AI-driven algorithm.

### Model Architecture

A deep learning framework combining convolutional and transformer layers was used (based on the architecture described in Annamalai N et al, “A Wide and Deep Transformer Neural Network for 12-Lead ECG Classification”. Computing in Cardiology, 2020). The convolutional layers initially extract features from ECG signals, and the transformer, with parameters like 256 embedding size, 8 heads, and 8 layers, captures sequential dependencies. Data loaders handle ECG data in stages (rest, exercise, recovery), applying filters and normalization by standard deviation. Training of the model involves a customized loss function, with hyperparameters of 500 sample rate, 0.2 dropout rate, 128 batch size and a 2-second window for ECG signals. The model uses an adaptive optimizer with specific learning rate adjustments, and training includes early stopping based on AUC-ROC performance.

The model's architecture employs a distinctive 2-second interval in ECG data processing, efficiently capturing the essential cardiac signatures. This method addresses the redundancy in ECG signals by randomly selecting varied segments in each training iteration. These brief intervals are as informative as longer recordings. Consequently, the model is exposed to a diverse range of signal patterns within individual ECG recordings, significantly enhancing its learning capacity and generalizability. By generating multiple predictions per subject from different ECG segments, the model offers a more robust assessment of cardiac health. This leads to a stable performance evaluation as averaging predictions across various segments provides a more reliable indicator of cardiac events, reducing variability and improving the overall accuracy of the model's assessments.

### Statistical Analysis

The study population was described by group (training/validation/test) using appropriate statistics. Each model was individually evaluated based on three metrics: the area under the receiver operating characteristic curve (AUROC), sensitivity, and specificity. For the sake of comparison, sensitivity and specificity were estimated at Youden's index, the point maximizing their sum for each model. Confidence intervals for the AUROCs were calculated using DeLong's method.

### Results:

In the disclosed study, it is clearly demonstrated that the model trained on the complete ECG dataset (encompassing portions of rest, stress, and recovery phases) exhibited enhanced performance as compared to training a model on the rest phase data alone.

Specifically, as shown in Table 2 below, the AUC for males improved from 0.62 to 0.73, while for females it increased from 0.686 to 0.776.

TABLE 2

| | | | |
|---|---|---|---|
| Group | AUC (95% CI) | Sensitivity | Specificity |

Model trained only on rest ECG part

| | | | | |
|---|---|---|---|---|
| Males | 0.62, | (0.597, 0.643) | 0.549 | 0.619 |
| Females | 0.686, | (0.634, 0.738) | 0.729 | 0.586 |
| Overall | 0.649, | (0.629, 0.669) | 0.534 | 0.665 |

Model trained on rest, stress and recovery parts

| | | | | |
|---|---|---|---|---|
| Males | 0.73, | (0.71, 0.751) | 0.745 | 0.65 |
| Females | 0.776, | (0.718, 0.833) | 0.771 | 0.732 |
| Overall | 0.753, | (0.734, 0.772) | 0.736 | 0.695 |

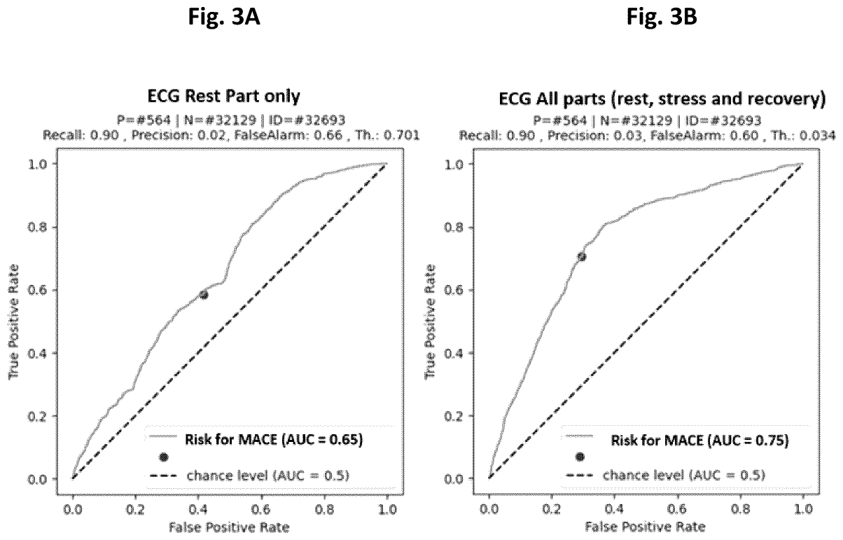

Furthermore, as shown in FIGS. 3A-B, there is a marked an improvement in the Area under the curve (AUC) from 0.65 when using only rest part data of ECG (FIG. 3A), to 0.75 when using all parts of the EET test (FIG. 3B). This improvement indicates a far more robust diagnostic ability of the model, as evidenced by heightened sensitivity and specificity across genders.

These results highlight the critical role of comprehensive ECG data in refining cardiovascular event predictions.

### Example 2—CAD Diagnosis and MACE Prediction Based on EET of a Patient Using a Trained Machine Learning Algorithm

An AI model was trained to analyze raw waveform data from EETs and predict a person's risk for a cardiovascular event within two years. Data was complied from 22,514 patients who underwent EETs at a large medical center over a span of 6 years, and the trained model was applied on the entire cohort (3-K fold cross-validation) (n=22,514, 30.4% females, median age 51, n=396 positives) for Major Adverse Cardiovascular Event (MACE) prediction and 60:20:20 training/validation/testing split correspondgly (n=3926, 12% females, median age 63, n=402 positives) for CAD detection. Collectively, as shown herein, the AI model, using the full ECG (three parts: rest, stress and recovery), yielded higher performance as compared to the rest-only ECG model, with increase of 0.31 points AUC for the true positive rate.

### Methods

### Study Design and Data

A retrospective study based on electronic medical records from a large tertiary healthcare center was performed. One outcome was the patient's occurrence of MACE (Major Adverse Cardiovascular Event) within 2 years from the EET (Exercise ECG Test) timing. Event was consider as one of the follow: ID (Ischemic heart Disease), CVA (cerebral vascular accident), PCI (Percutaneous coronary intervention) or Mortality. Another was existence of moderate to severe myocardial ischemia, defined as over 5% myocardial ischemia as viewed in a SPECT-based Heart Mapping test.

All patients ≥18 with available digital 12 lead Exercise ECG performed in the institution over 6-years was used to create the dataset. Patients missing measurements in the EET report or with incomplete or corrupted ECG waveforms were excluded from the study. All ECGs were acquired as digital standard 12-lead ECGs using Norav Medical ECG devices. The final cohort was divided into 3 subsets, using outcome-stratified random sampling for a cross-validation strategy.

The primary analysis was binary classification using the 12-leads ECG recording, sub-group analysis by age and sex also was performed.

### Overview of the AI Model

### Data Pre-Processing

In the preprocessing phase, raw ECG waveform data extracted from rdt files (Norav raw ECG data format), which are then converted into matrix (numpy format), each cell in the matrix represent voltage over time. Eventually a csv file (table) was created for each test contains 12 columns represents the 12 leads and X amount of raws based on the recording timing, for example 10 seconds will contains 5000 raws since the sample rate is 500. For each sample, a quality checks for constancy and completeness was performed. The ECG data was segmented into distinct phases: rest, peak exercise, and recovery.

ECG data segmentation into rest, peak exercise, and recovery phases is driven by clinical rationale. The rest phase establishes baseline cardiac function, key for spotting irregularities. The peak exercise phase, when the heart is under maximum stress, uncovers potential ischemic issues not visible at rest. The recovery phase assessment is crucial for evaluating the heart's return to baseline, providing insights into overall cardiac health. This targeted analysis across different stress levels on the heart is essential for a comprehensive cardiovascular assessment, aiding in the prediction of major adverse events.

Raw waveform underwent a few stages to reduce noise levels and provide uniformity intra-patient and inter-patient. Data provided as integer was converted into floating-point numbers and divided by 1,000 to bring it to known scales. Only leads aVR, aVF, V1-V6 were taken to reduce data redundancy. A 7^{th} order Butterworth Bandpass Filter 1-30 Hz was applied twice (back and forth, as is common with IIR filters) to reduce motion, breathing, sweat, muscles and powerline noises. Signal is decimated by a factor of 5 to reduce output sizes and segmented into R-R-R intervals using V5/V6 R-peak detection. Resultant intervals were interpolated using cubic spline to 2.5 seconds (on 100 Hz, equals 250 sample points) as to provided consistency between beats of variable heart rates. Data is saved as 16-point float using a Gzip-compressed parquet file.

A custom function is them applied to split the dataset into several folds or training/validation/testing split, ensuring balanced representation of event and non-event cases by use of label-gender-age bin stratification (age bins: 0-40, 40-60, 60-80, 80-100). This step includes options for random shuffling. Finally, the script merges the processed data with fold information.

The MACE cohort was split into 3 folds for cross validation strategy.

The CAD cohort was split into training/validation/testing.

This detailed preprocessing ensures the uniformity, integrity and usability of the ECG data for developing our AI-driven algorithm.

### Model Architecture

A Maximum Mean Discrepency Variational Autoencoder (MMD-VAE or InfoVAE) deep learning foundational model was developed, that takes R-R-R segments and outputs embeddings of 48 latent dimensions, using MMD and Mean Squared Error losses functions. Embeddings are then fed into transformer-encoder model developed using a learned/trained rest/exercise/recovery section embedding. A fixed class token is added prior to the embeddings for classification. The transformer block uses 4 encoders, 2 attention heads and 64 feed-forward dimension. The transformer block output for the classification token is fed into a 16-dimension fully connected layer with a ReLU activation and then to a 1-dimension output fully connected layer. Loss used was a Sigmoid Focal Loss with alpha=0.1, to assist with class imbalance. Optimizer used is AdamW with AMSGrad and Weight Decay of 0.2, at learning rate 0.0001. Validation set was evaluated at end of each epoch, and early stop was defined as 30 epochs with no improvement to validation loss. Evaluations was performed based on model weights with lowest validation loss.

Data was fed in the form of embeddings with a variable length due to different recording lengths. A linear regression was performed for each embedding dimension over a section. If the regression was found significant (p-value<0.05), residuals were calculated and Z-scored. Residual embeddings over 2SD were discarded to improve consistency and reduce noise. If the regression was not significant, the slope was assumed to be zero, and only Z-scoring was performed prior to >2SD embeddings outlier discarding. Alongside the embeddings, an input mask was given to denote variable recording lengths, and fed into the attention blocks for improved performance and accuracy as is common with this architecture. Target was changed between MACE and CAD, based on the dataset, producing different model weights.

### Statistical Analysis

Each model was individually evaluated based on three metrics: the area under the receiver operating characteristic curve (AUROC), sensitivity, and specificity. For the sake of comparison, sensitivity and specificity were estimated at Youden's index, the point maximizing their sum for each model. Confidence intervals for the AUROCs were calculated using DeLong's method.

### Results

In the study, the model trained on the complete ECG dataset (encompassing rest, stress, and recovery phases) showed enhanced performance compared to training on the rest phase alone.

In the MACE dataset specifically, the AUC for males improved from 0.658 to 0.679, while for females it increased from 0.644 to 688, as shown in Table 3 below.

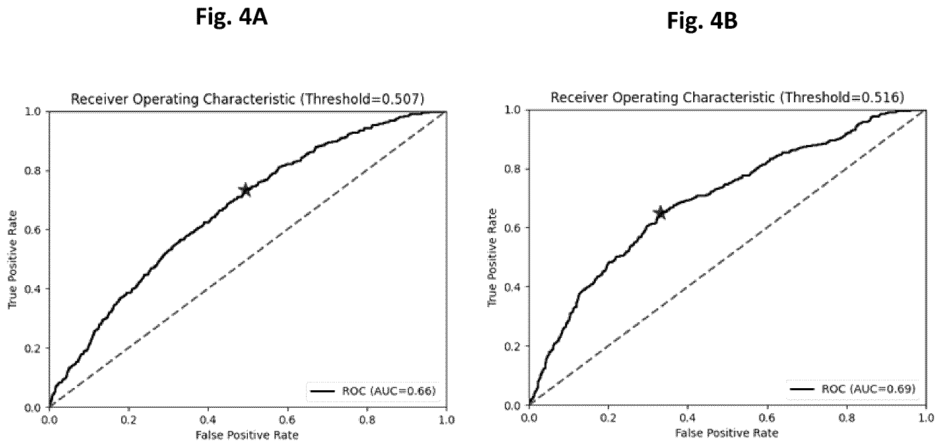

Overall an improvement in the AUC from 0.664 when using rest part only to 0.695 when using all parts of the test was demonstrated, as shown in FIGS. 4A-B.

TABLE 3
MACE Prediction

| | | | |
|---|---|---|---|
| Group | AUC (95% CI) | Sensitivity | Specificity |

Model trained only on rest ECG part

| | | | | |
|---|---|---|---|---|
| Males | 0.66, | (0.631, 0.685) | 0.737 | 0.488 |
| Females | 0.64, | (0.566, 0.723) | 0.717 | 0.548 |
| Overall | 0.66, | (0.639, 0.69) | 0.735 | 0.506 |

Model trained on rest, stress and recovery parts

| | | | | |
|---|---|---|---|---|
| Males | 0.68, | (0.651, 0.707) | 0.663 | 0.627 |
| Females | 0.69, | (0.607, 0.769) | 0.543 | 0.771 |
| Overall | 0.695, | (0.668, 0.721) | 0.649 | 0.67 |

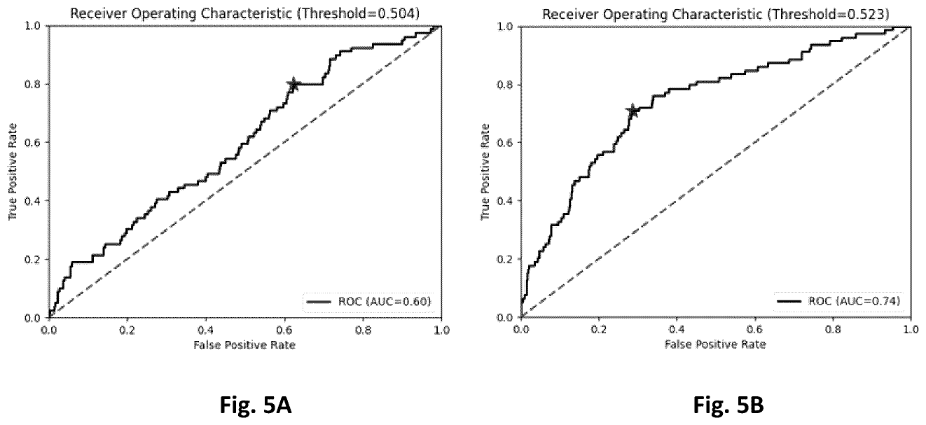

For the CAD dataset, AUC for males improved from 0.55 to 0.7, 0.583 to 0.826 for females, and from 0.598 to 0.742 overall, as shown in FIGS. 5A-5B. This improvement indicates a more robust diagnostic ability of the model, as evidenced by heightened sensitivity and specificity across genders.

Collectively, the results demonstrate the critical role of comprehensive ECG data in refining cardiovascular event predictions and diagnosis of myocardial ischemia.

### Example 3—Preprocessing of Raw EET and Segmentation Thereof for Use in Training and Applying AI Algorithms for Prediction of MACE and/or Detection of CAD

In order to overcome challenges with interpretation of EET tests due to, for example, signal noise patterns (increasing with strain, breathing, sweating and electrode movement) a preprocessing stage is applied to each recording.

To this aim, Raw files containing 4-digits integers including measured voltage levels in 12-Lead ECG (I, II, III, aVR, aVF, aVL, V1, V2, V3, V4, V5, V6) are loaded and converted into 32-bit floating-point arrays and divided by a factor of 1,000, to bring them to micro-volt scale. Only several leads (for example, 8, such as, aVR, aVF, V1, V2, V3, V4, V5, V6) may be selected out of the input, as the remaining ones can be computed. A 7-th order Butterworth 1-30 Hz Bandpass filter is applied to remove noise components, such as baseline drift, breathing and muscle activity. Then, the signal is decimated from the recorded 500 Hz sampling rate, by a factor of five to bring the resulting sampling rate to 100 Hz. Each recording is segmented into two sections—exercise and recovery, where the exercise segment is the one defined by a Bruce Protocol.

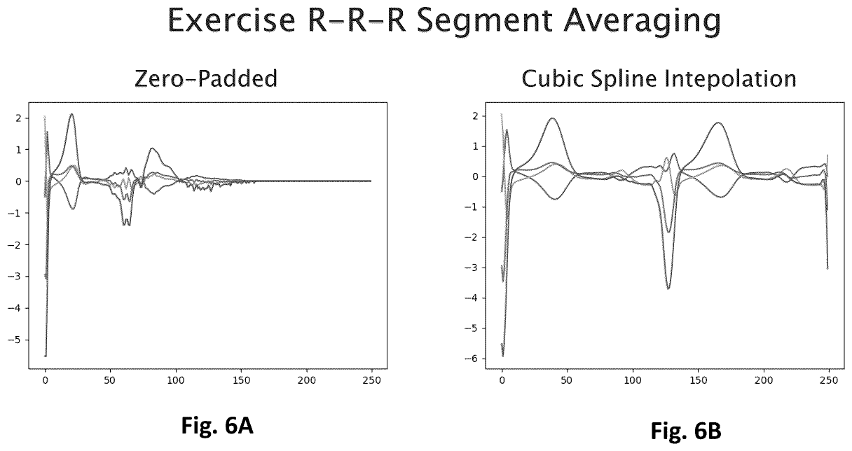

To generate a more standardized input for the machine learning models, each patient's naturally forming segments that vary with heart rate are utilized. To this aim, the R-peaks of the ECG QRS complex are identified along V3-V6 or V5-V6 peaks. The difference between R-peak locations are used to calculate the variance across each of the selected leads. The lead with the least variance is considered the most consistent and is used for a “R-R-R” segmentation across all other leads. R-R-R segments contain all necessary information about momentary heart activity, including a complete QRS complex in the middle, P-wave, T-wave and other potential cardiac electrical activity. Each segment begins and ends in R-peaks of adjacent QRS complexes. As each segment length varies, a cubic spline interpolation may be used to stretch the input segment to standardized 250 samples, corresponding to 2.5 seconds in cases where no interpolation is necessary in low heart rates. This approach allows to more accurately compare ECG components in various heart beats. The results are presented in FIGS. 6A-B, showing line graphs of Averaging 100 R-R-R segments from rest ECG. FIG. 6A shows zero-padded segments averaged. The appearance of dual-peaks and the destruction of late-segment activity can be noted. FIG. 6B shows line graphs of averaging after applying cubic spline interpolation. As shown, averaging forms a more coherent activity throughout the segment with noticeable ECG components.

The preprocessed data may then be further used in the training and detection algorithms, as detailed above. Each input recording is saved as two parquet files containing 16-bit floating point data after preprocessing. The shape of the output file is (250*N, 8) where 250 stems from interpolation parameter, 8 is the number of leads after reduction and N is the amount of R-R-R segments found in the signal. Each of the three recordings corresponds with exercise and recovery sections.

What is claimed is:

1. A computer implemented method for prediction of major adverse cardiovascular events (MACE) and/or detection of coronary artery disease (CAD) in a subject, the method comprising:
receiving exercise electrocardiogram (ECG) test (EET) data of the subject;
inputting waveform data from the EET data to a predictive machine learning (ML) algorithm, wherein the machine learning algorithm was trained on a dataset comprising data patterns of R-R-R segments of EET data obtained from a plurality of subjects and correlated with the presence or absence a coronary artery disease in each of said plurality of subjects;
outputting by the machine learning algorithm, a MACE risk score and/or a CAD probability score for the patient, based on the inputted data from the EET data for the subject;
to thereby provide prediction of CAD or MACE in the patient.

2. The method according to claim 1, wherein the raw data is preprocessed to remove noise components, to standardize the data and/or to segment into at least exercise and recovery sections.

3. The method according to claim 1, wherein the R-R-R segments comprise information regarding momentary heart activity, comprising a complete QRS complex.

4. The method according to claim 1, wherein the EET test comprises recording obtained from single-lead, multi-lead, a 12-lead test, or any combinations thereof.

5. The method according to claim 1, wherein data obtained from at least a portion of EET leads is utilized for the determination of the R-R-R segments.

6. The method according to claim 1, wherein data obtained from leads V5-V6 is used for selecting a lead having least variance between R peaks, for the R-R-R segmentation.

7. The method according to claim 1, wherein R-R-R segmentation of R-peaks of QRS complex are determined for at least a portion of selected leads, based on:
determining differences between R-peak locations for each of the selected leads;
calculating variance in R-peak locations across each of the selected leads; and
selecting the lead with the least variance between the peaks for R-R-R segmentation.

8. The method according to claim 1, further comprising standardizing length of the R-R-R segments.

9. The method according to claim 1, further comprising applying a classification transformer.

10. The method according to claim 1, wherein the EET data comprises raw waveform EET data of at least a portion of at least three phases of EET.

11. The method according to claim 10, the at least three phases of the EET comprise a rest phase, a stress phase and a recovery phase.

12. The method according to claim 1, wherein the dataset is split into a plurality of groups, to enhance balanced representation of cardiac related events, and non-related events.

13. The method according to claim 1, wherein the period of time for MACE prediction is for 6 months or more, from performing the EET.

14. The method according to claim 1, wherein the EET data further comprises oxygen consumption of the subject, heart rate, blood pressure, or any combinations thereof.

15. The method according to claim 1, wherein the method further comprises providing a therapy recommendation to the patient, based on the MACE risk score and/or the CAD probability score output.

16. The method according to claim 15, wherein the therapy comprises: a pharmaceutical therapy, behavioral therapy, a surgical therapy, or any combinations thereof.

17. A system for prediction of MACE and/or detection of coronary artery disease CAD in a subject, the system comprising a processor configured to execute the method of claim 1.

18. The system according to claim 17, further comprising or communicatively associated with one or more of: an ECG unit, a display, a user interface, a memory, a local server, a remote server, a communication unit, a database, or any combination thereof.

19. A non-transitory computer-readable medium storing processor executable instructions on a computing device, when executed by a processor, the processor executable instructions causing the processor to perform the method of claim 1.
